(19) United States
(12) Patent Application Publication
(10) Pub. No.: US 2006/0241651 A1
(43) Pub. Date: Oct. 26, 2006

(54) Surgical port device and associated method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/387,522

(22) Filed: Mar. 23, 2006

Related U.S. Application Data
(60) Provisional application No. 60/674,041, filed on Apr. 22, 2005.

(51) Int. Cl. A61F 11/00

(52) U.S. Cl. 606/108; 600/192

(57) ABSTRACT

A surgical method comprises inserting a distal end portion of a surgical instrument through a natural body opening of a patient into a natural body cavity of the patient, using the surgical instrument to form a temporary artificial opening through a wall of an organ defining the natural body cavity, and providing a surgical device including a membrane, a plurality of tubular members traversing the membrane, and means for attaching the membrane to a wall of an organ so that the tubular members traverse a perforation in the wall. The port device is inserted through the natural body opening into the natural body cavity, and subsequent to the inserting of the port device, the port device is disposed in the artificial opening to keep the same open.

### CROSS-REFERENCE TO RELATED APPLICATION

This application claims the benefit of U.S. Provisional Patent Application No. 60/674,041 filed Apr. 22, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There are some potential problems with the procedures, such as the difficulty in forming a fluid tight closure of the perforation formed in the wall of the hollow internal body organ.

### OBJECTS OF THE INVENTION

It is an object of the present invention to provide improvements on the afore-described surgical procedures.

It is another object of the present invention to provide a method and/or an associated device for keeping a passageway open in an internal hollow organ.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical port device in accordance with the present invention comprises a membrane, a plurality of tubular members traversing the membrane, and means for attaching the membrane to a wall of an organ so that the tubular members traverse or pass through a perforation in the wall.

Pursuant to another feature of the present invention, the attaching means includes at least one annular balloon member. The balloon member may be one of two annular balloon members of substantially the same size aligned with one another to clamp the organ wall in sandwich fashion about the perforation. The balloon members are located along a periphery of the membrane. The balloon member or members are provided an inflation tube for enabling an introduction of a fluid into the balloon member(s) to expand the same from a collapsed configuration to an inflated configuration.

Pursuant to a further feature of the present invention, the tubular members are each provided internally with a sealing or valve element.

Pursuant to an additional feature of the present invention, respective balloon members constitute the tubular members. A fluid guide is operatively connected to the tubular balloon members for guiding a fluid thereto to inflate the tubular balloon members from a collapsed configuration to an expanded use configuration.

An ancillary tube may extend from one side of the membrane to an opposite side thereof, where the tube is provided with a coupling element at one end for connecting the tube to a source of pressurized gas.

The membrane may be provided with an element made of a resilient shape-memory material. The element may be a radial rib (of a plurality of radial ribs) or a ring preferably disposed proximate to the periphery of the membrane.

A surface of at least one of the balloon or a clamping element may be provided with a layer of an dormant adhesive substance that is activated by the application of a predetermined form of energy. The clamping element may be an annular disk or flange element that cooperates with the annular balloon member to anchor the port device to the organ wall.

A surgical method comprises, in accordance with the present invention, (a) inserting a distal end portion of a surgical instrument through a natural body opening of a patient into a natural body cavity of the patient, (b) using the surgical instrument to form a temporary artificial opening through a wall of an organ defining the natural body cavity, (c) providing a surgical port device including a membrane, a plurality of tubular members traversing the membrane, and means for attaching the membrane to a wall of an organ so that the tubular members traverse a perforation in the wall, (d) inserting the port device through the natural body opening into the natural body cavity, and (e) subsequent to the inserting of the port device, disposing the port device in the artificial opening to keep the same open.

The surgical method may further comprise, in accordance with the present invention, inserting a distal end portion of a medical instrument through the natural body opening, the natural body cavity, one of the tubular members, and the artificial opening into an internal space inside the patient after the disposing of the port device in the artificial opening.

The surgical method may additionally comprise introducing a pressurized gas into the internal space via an elongate tube communicating with the internal space via the port device.

Where the surgical port device further includes (i) an annular balloon attached to the membrane along a periphery thereof and (ii) a clamping element attached to the membrane along the periphery, the clamping element being at least partially aligned and coextensive with the balloon, the method further comprises (f) inserting one of the balloons in a deflated configuration and the clamping element through the artificial opening and (g) thereafter inflating the balloon to sandwich a portion of the wall between the inflated balloon and the clamping element.

Where one of the clamping element and the balloon is provided with a layer of an activatable adhesive, the method additionally comprises directing a predetermined form of energy towards a surface of the one of the disk and the balloon in contact with the wall of the organ to activate the adhesive.

### BRIEF DESCRIPTION OF THE DRAWING

FIG. 1 is a schematic perspective view of a surgical port device in accordance with the present invention, showing the port device attached to an organ wall.

FIG. 2 is a schematic perspective view of another surgical port device in accordance with the present invention, showing the port device attached to an organ wall.

FIG. 3 is a partial cross-sectional view through the port device of FIG. 1 or 2, showing structural details of the port devices.

FIG. 4 is a schematic cross-sectional view similar to FIGS. 1 and 2, showing a step in a surgical method using the surgical port device of FIG. 1.

### DETAILED DESCRIPTION

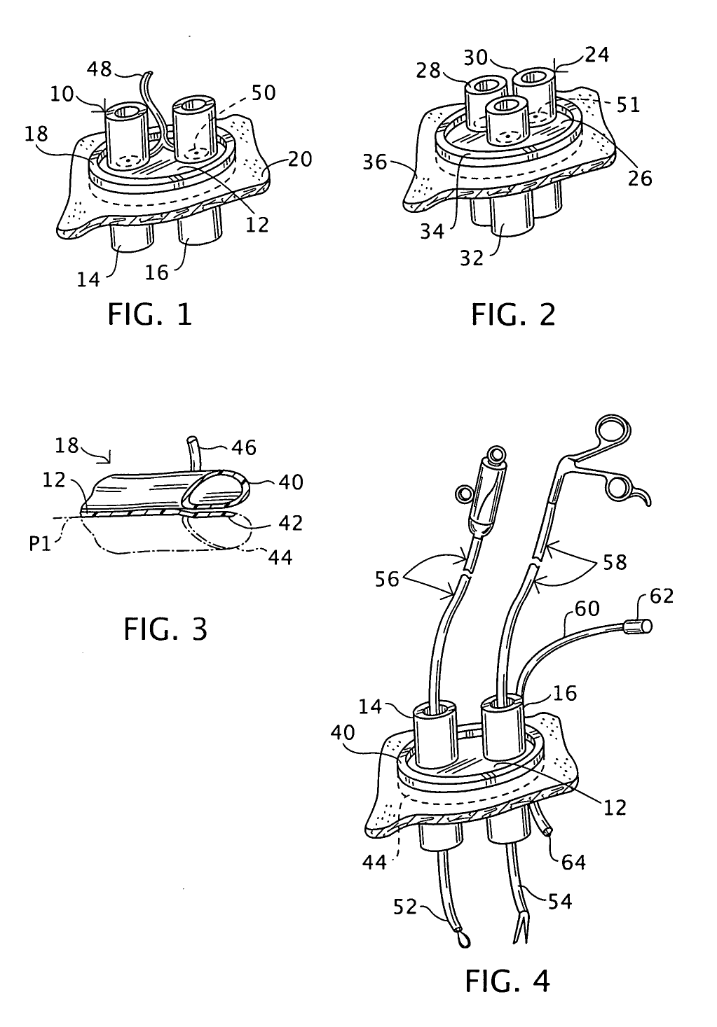

FIG. 1 illustrates a surgical port device 10 particularly for use in a trans-organ surgical procedure as described in U.S. Pat. Nos. 5,297,536 and 5,458,131 (both incorporated by reference herein). Port device 10 comprises a membrane 12, two tubular members 14 and 16 traversing the membrane, and components 18 for attaching the membrane to a wall 20 of an organ so that the tubular members traverse or pass through a perforation or opening (not shown) artificially formed in the organ wall.

FIG. 2 illustrates a modified surgical port device 24 that comprises a membrane 26, three tubular members 28, 30, 32 traversing the membrane, and components 34 for attaching the membrane to a wall 36 of an organ so that the tubular members traverse or pass through a perforation or opening (not shown) artificially formed in the organ wall.

As depicted in FIG. 3, attaching components 18 (or 34) include at least one annular balloon member 40. Attaching components 18 (or 34) additionally include a clamping member in the form of an annular disk 42 or a balloon 44. Clamping member 42 or 44 and balloon 40 may be substantially coextensive in a plane P1. Clamping members 42 and 44 function to clamp organ wall 20 (or 36) in sandwich fashion about perforation or incision 22 (or 38).

Balloon member 40 and clamping member 42 or 44 are located along a periphery of membrane 12 (or 26). Balloon member 40 and optionally balloon member 44 are provided with at least one an inflation tube 46 for enabling an introduction of a pressurizing fluid into the balloon member(s) to expand the same from a collapsed insertion configuration to an inflated use configuration.

Port devices 10 and 24 may each be deployed by disposing it in a folded or collapsed configuration (not shown) in a distal end portion of a delivery tube (not shown), inserting the distal end portion of the delivery tube into the patient through a natural body opening such as the mouth, the vaginal orifice, the anus, or the urethra orifice, and ejecting the port device from the distal end of the delivery tube. In that case, tubular members 14, 16, 28, 30, 32 may take the form of balloons that are inflated into a cylindrical form upon the ejection of the collapsed or folded port device 10, 24 from the distal end of the delivery tube. One or more inflation tubes 48 may be provided for delivering a pressurization fluid such as carbon dioxide or saline solution from a reservoir to tubular members 14, 16, 28, 30, 32 for expanding the same. Alternatively or additionally, tubular members 14, 16, 28, 30, 32 as well as membranes 12 and 26 may be provided with shape-memory elements such as longitudinal or radial ribs and rings (not shown) for inducing a reformation of the expanded use configurations of port devices 10 and 24 shown in FIGS. 1 and 2.

Each tubular member 14, 16, 28, 30, 32 may be provided internally with a sealing or valve element 50, 51 forming an effectively air tight seal with an outer surface of a flexible shaft 52, 54 of a surgical or medical instrument 56, 58 inserted through tubular members 14, 16, 28, 30, 32 during a trans-organ surgical procedure as described in U.S. Pat. Nos. 5,297,536 and 5,458,131.

An ancillary tube 60 may extend from one side of membrane 12 or 26 to an opposite side thereof, where the tube is provided at a proximal end with a coupling element 62 for connecting the tube to a source of pressurized gas (not illustrated). At a distal end 64, tube 60 has an outlet (not separately enumerated) for dispensing gas into an internal space such as the abdominal cavity to maintain pneumoperitoneum during a trans-organ procedure.

A surface of clamping element 42 or 44 may be provided with a layer of a dormant adhesive substance that is activated by the application of a predetermined form of energy (electromagnetic radiation such as microwave, infrared, radiowave or ultrasound).

A surgical method comprises, as described in U.S. Pat. Nos. 5,297,536 and 5,458,131, inserting a distal end portion of a surgical instrument through a natural body opening of a patient into a natural body cavity of the patient, and using the surgical instrument to form a temporary artificial opening through a wall of an organ defining the natural body cavity. In carrying out the method, surgical port device 10 or 24 is inserted through the natural body opening into the natural body cavity. Subsequently, port device 10 or 24 is inserted or placed in the artificial opening (perforation, incision) to keep the same open.

Distal end portions of multiple surgical or medical instruments 56, 58 are inserted through the natural body opening, the natural body cavity, respective ones of the tubular members 14, 16, 28, 30, 32, and the artificial opening into an internal space (e.g., abdominal cavity) inside the patient after the disposing of port device 10, 24 in the artificial opening.

The surgical method may additionally comprise introducing a pressurized gas into the internal space via elongate tube 60 that communicates with the internal space via the port device 10, 24.

The method further comprises inserting coupling element 42 or 44 in a deflated or folded configuration through the artificial opening and thereafter expanding the clamping element 42, 44 to sandwich a portion of the organ wall between the inflated balloon 40 and the clamping element 42 or 44.

Ports devices 10, 24 may be marketed as components of surgical kits that include medical or surgical operating instruments 56, 58, etc.

Although the invention has been described in terms of particular embodiments and applications, one of ordinary skill in the art, in light of this teaching, can generate additional embodiments and modifications without departing from the spirit of or exceeding the scope of the claimed invention. For example, a port device as described herein may include barbs or hooks along a surface that contacts an organ wall, for assisting in maintaining the port element in contact with the organ wall. Accordingly, it is to be understood that the drawings and descriptions herein are profferred by way of example to facilitate comprehension of the invention and should not be construed to limit the scope thereof.

What is claimed is:

1. A surgical port device comprising:
a membrane;
a plurality of tubular members traversing said membrane; and
means for attaching said membrane to a wall of an organ so that said tubular members traverse or pass through a perforation in said wall.

2. The surgical port device defined in claim 1 wherein said means for attaching includes at least one annular balloon member.

3. The surgical port device defined in claim 2 wherein said balloon member is one of two annular balloon members, said balloon members being of substantially the same size and are aligned with one another to clamp said wall in sandwich fashion about said perforation, said balloon members being located along a periphery of said membrane.

4. The surgical port device defined in claim 3 wherein said tubular members are each provided internally with a sealing or valve element.

6. The surgical port device defined in claim 2 wherein said balloon member is provided an inflation tube for enabling an introduction of a fluid into said balloon member to expand same from a collapsed configuration to an inflated configuration.

5. The surgical port device defined in claim 1 wherein said tubular members are each provided internally with a sealing or valve element.

7. The surgical port device defined in claim 1 wherein said tubular members are constituted by respective balloon members, further comprising means operatively connected to said balloon members for guiding a fluid thereto to inflate said balloon members from a collapsed configuration to an expanded use configuration.

8. The surgical port device defined in claim 1, further comprising a tube extending from one side of said membrane to an opposite side thereof, said tube being provided with a coupling element at one end for connecting said tube to a source of pressurized gas.

9. The surgical port device defined in claim 1 wherein said membrane is provided with an element made of a resilient shape-memory material.

10. The surgical port device defined in claim 1 wherein a surface of at least one of said disk and said balloon is provided with a layer of an dormant adhesive substance that activated by the application of a predetermined form of energy.

11. The surgical port device defined in claim 1 wherein said membrane, said tubular members, and said means for attaching are all made of a flexible material enabling an insertion of the device in a collapsed compact configuration.

12. A surgical method comprising:
inserting a distal end portion of a surgical instrument through a natural body opening of a patient into a natural body cavity of the patient;
using said surgical instrument to form a temporary artificial opening through a wall of an organ defining said natural body cavity;
providing a surgical port device including a membrane, a plurality of tubular members traversing said membrane, and means for attaching said membrane to a wall of an organ so that said tubular members traverse a perforation in said wall;
inserting said port device through said natural body opening into said natural body cavity; and
subsequent to the inserting of said port device, disposing said port device in said artificial opening to keep the same open.

13. The surgical method defined in claim 12, further comprising, after the disposing of said port device in said artificial opening, inserting a distal end portion of a medical instrument through said natural body opening, said natural body cavity, one of said tubular members, and said artificial opening into an internal space inside the patient.

14. The surgical method defined in claim 13, further comprising introducing a pressurized gas into said internal space via an elongate tube communicating with said internal space via said port device.

15. The surgical method defined in claim 12 wherein said surgical port device further includes an annular balloon attached to said membrane along a periphery thereof, said surgical port device further including a clamping element attached to said membrane along said periphery, said clamping element being at least partially aligned and coextensive with said balloon, further comprising: inserting one of said balloon in a deflated configuration and said clamping element through said artificial opening; and thereafter inflating said balloon to sandwich a portion of said wall between the inflated balloon and said clamping element.

16. The surgical method defined in claim 15 wherein said one of said clamping element and said balloon is provided with a layer of an activatable adhesive, further comprising directing a predetermined form of energy towards a surface of said one of said clamping element and said balloon in contact with said wall of said organ to activate said adhesive.

17. A surgical kit comprising:
at least one surgical instrument having an elongate flexible shaft; and
a port device including:
a membrane;
a plurality of tubular members traversing said membrane; and
means for attaching said membrane to a wall of an organ so that said tubular members traverse or pass through a perforation in said wall

18. The surgical kit defined in claim 17 wherein said means for attaching includes at least one annular balloon member.

19. The surgical kit defined in claim 17 wherein said tubular members are each provided internally with a sealing or valve element.

20. The surgical kit defined in claim 17 wherein said tubular members are constituted by respective balloon members, further comprising means operatively connected to said balloon members for guiding a fluid thereto inflate said balloon members from a collapsed configuration to an expanded use configuration.

21. The surgical kit defined in claim 17, further comprising a tube extending from one side of said membrane to an opposite side thereof, said tube being provided with a coupling element at one end for connecting said tube to a source of pressurized gas.

22. The surgical kit defined in claim 17, wherein said membrane, said tubular members, and said means for attaching are all made of a flexible material enabling an insertion of the device in a collapsed compact configuration.
